(19) United States
(12) Patent Application Publication
(10) Pub. No.: US 2024/0415834 A1
(43) Pub. Date: Dec. 19, 2024

(54) LURBINECTEDIN AND ATEZOLIZUMAB COMBINATIONS

(71) Applicant: PHARMA MAR, S.A., Madrid (ES)

(72) Inventors: Luis Gonzaga Paz-Ares Rodriguez, Madrid (ES); Santiago Ponce Aix, Madrid (ES); Salvador Fudio Muñoz, Madrid (ES)

(21) Appl. No.: 18/703,491

(22) PCT Filed: Nov. 8, 2022
(86) PCT No.: PCT/EP2022/081155

§ 371 Date: Apr. 22, 2024

(30) Foreign Application Priority Data
Nov. 8, 2021 (EP) 21383013.6

(51) Int. Cl. A61K 31/4995; A61K 38/19; A61K 39/00; A61K 39/395; A61P 35/00

(52) U.S. Cl. CPC A61K 31/4995; A61K 38/193; A61K 39/3955; A61P 35/00; A61K 2039/545

(57) ABSTRACT

Described are combination therapies for the treatment of small cell lung cancer comprising lurbinectedin and atezolizumab.

### FIELD OF THE INVENTION

The present invention relates to therapeutic treatment of cancers, particularly dosing schedules useful in the treatment of cancer. In particular, the present invention relates to combination therapy using lurbinectedin and atezolizumab.

### BACKGROUND OF THE INVENTION

Atezolizumab (MPDL3280A) is a humanized IgG1 monoclonal antibody consisting of two heavy chains (448 amino acid residues each) and two light claims (214 amino acid residues each) and is produced in Chinese hamster ovary cells. Atezolizumab targets human PD-L1 and inhibits its interaction with its receptors, programmed cell death protein 1 (PD-1) and B7.1 (CD80, B7-1).

Both of these interactions are reported to provide inhibitory signals to T cells. Atezolizumab is approved in USA and Europe for the treatment of patients with metastatic NSCLC whose disease progressed during or following platinum-containing chemotherapy.

Lurbinectedin, also known as PM01183 and initially called tryptamicidin, is a synthetic tetrahydropyrrolo[4,3,2-de]quinolin-8(1H)-one alkaloid analogue with antineoplastic activity, and the subject of WO 03/01427. Lurbinectedin is a selective inhibitor of oncogenic transcription, induces DNA double-strand break generating apoptosis, and modulates the tumour microenvironment. For example, by inhibiting active transcription in tumour-associates macrophages, lurbinectedin downregulates IL-6, IL-8, CCL2, and VEGF.

The chemical structure of lurbinectedin is represented as follows:

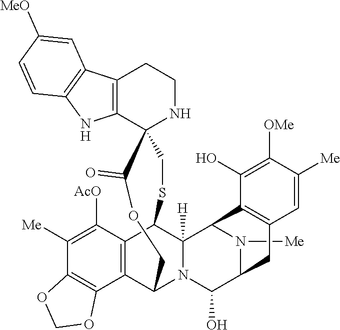 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

Lurbinectedin has demonstrated highly potent in vitro activity against solid and non-solid tumour cell lines as well as significant in vivo activity in several xenografted human tumour cell lines in mice, such as those for breast, kidney and ovarian cancer. It is a selective inhibitor of the oncogenic transcription programs on which many tumours are particularly dependent. Together with its effect on cancer cells, lurbinectedin inhibits oncogenic transcription in tumour-associated macrophages, downregulating the production of cytokines that are essential for the growth of the tumour. Transcriptional addiction is an acknowledged target in those diseases, many of them lacking other actionable targets.

There is a need for further effective cancer therapies.

### SUMMARY OF THE INVENTION

The present inventors have surprisingly determined dosing regimens of lurbinectedin and atezolizumab effective in the treatment of small cell lung cancer.

Accordingly, in an aspect of the present invention there is provided lurbinectedin and atezolizumab for use in the treatment of cancer, wherein said treatment comprises administering to a patient in need thereof a combination of lurbinectedin and atezolizumab; wherein lurbinectedin is administered at a dose of 3.2 mg/m2 and atezolizumab is administered at a dose of 1200 mg; and wherein lurbinectedin and atezolizumab are administered on day 1 of a 21 day cycle.

In a further aspect there is provided lurbinectedin and atezolizumab for use in the treatment of small cell lung cancer, wherein said treatment comprises administering to a patient in need thereof a combination of lurbinectedin and atezolizumab; optionally wherein the small cell lung cancer is extensive stage small cell lung cancer (ES-SCLC).

In a further aspect there is provided a method of treatment of cancer, the method comprising administering to a patient in need thereof a combination of lurbinectedin and atezolizumab; wherein lurbinectedin is administered at a dose of 3.2 mg/m2 and atezolizumab is administered at a dose of 1200 mg; and wherein lurbinectedin and atezolizumab are administered on day 1 of a 21 day cycle.

In a further aspect there is provided a method of treatment of small cell lung cancer, the method comprising administering a combination therapy of lurbinectedin and atezolizumab; optionally wherein the small cell lung cancer is extensive stage small cell lung cancer (ES-SCLC).

In a further aspect there is provided the use of lurbinectedin and atezolizumab in the manufacture of a medicament for the treatment of cancer, wherein said treatment comprises administering to a patient in need thereof a combination of lurbinectedin and atezolizumab; wherein lurbinectedin is administered at a dose of 3.2 mg/m2 and atezolizumab is administered at a dose of 1200 mg; and wherein lurbinectedin and atezolizumab are administered on day 1 of a 21 day cycle.

In a further aspect there is provided the use of lurbinectedin and atezolizumab in the manufacture of a medicament for the treatment of small cell lung cancer, wherein said treatment comprises administering to a patient in need thereof a combination of lurbinectedin and atezolizumab; optionally wherein the small cell lung cancer is extensive stage small cell lung cancer (ES-SCLC).

In a further aspect there is provided a method of inhibiting cancer cell growth, comprising contacting cancer cells with a combination of lurbinectedin and atezolizumab; wherein said cancer cells are small cell lung cancer cells; optionally extensive stage small cell lung cancer (ES-SCLC) cells.

In a further aspect there is provided a method of treatment of cancer, the method comprising administering a combination therapy of lurbinectedin and atezolizumab to a patient in need thereof, wherein lurbinectedin is administered at a dose of between 2.5 to 3.2 mg/m², thereby treating the cancer. In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided the use of lurbinectedin in the manufacture of a medicament for the treatment of cancer, wherein said treatment comprises administering a combination therapy of lurbinectedin and atezolizumab to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided the use of atezolizumab in the manufacture of a medicament for the treatment of cancer, wherein said treatment comprises administering a combination therapy of lurbinectedin and atezolizumab to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided the use of lurbinectedin and atezolizumab in the manufacture of a medicament for the treatment of cancer, wherein said treatment comprises administering a combination therapy of lurbinectedin and atezolizumab to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided lurbinectedin for use in the treatment of cancer, wherein said treatment comprises administering a combination therapy of lurbinectedin and atezolizumab to a patient in need thereof, wherein lurbinectedin is administered at a dose of between 2.5 to 3.2 mg/m². In a particular embodiment, atezolizumab is administered at a dose of 840 mg to 1680 mg.

In a particular embodiment, lurbinectedin is administered at a dose of 3.2 mg/m² and atezolizumab is administered at a dose of 1200 mg.

In a further aspect there is provided atezolizumab for use in the treatment of cancer, wherein said treatment comprises administering a combination therapy of lurbinectedin and atezolizumab to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided lurbinectedin and atezolizumab for use in the treatment of cancer, wherein said treatment comprises administering a combination therapy of lurbinectedin and atezolizumab to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided lurbinectedin for use in the treatment of cancer, wherein in said treatment lurbinectedin is administered in combination with atezolizumab to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided atezolizumab for use in the treatment of cancer, wherein in said treatment atezolizumab is administered in combination with lurbinectedin to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided lurbinectedin and atezolizumab for use in the treatment of cancer, wherein in said treatment lurbinectedin is administered in combination with atezolizumab to a patient in need thereof, wherein lurbinectedin is administered at a dose of between 2.5 to 3.2 mg/m². In a particular embodiment, atezolizumab is administered at a dose of 840 mg to 1680 mg. In a particular embodiment, lurbinectedin is administered at a dose of 3.2 mg/m² and atezolizumab is administered at a dose of 1200 mg.

In a further aspect there is provided a method of treatment of small cell lung cancer, the method comprising administering a combination therapy of lurbinectedin and atezolizumab to a patient, preferably a human patient, in need thereof, thereby treating the small cell lung cancer. The small cell lung cancer may be extensive stage small cell lung cancer (ES-SCLC).

In a further aspect there is provided the use of lurbinectedin in the manufacture of a medicament for the treatment of small cell lung cancer, wherein said treatment comprises administering a combination therapy of lurbinectedin and atezolizumab to a patient in need thereof. The small cell lung cancer may be ES-SCLC.

In a further aspect there is provided the use of atezolizumab in the manufacture of a medicament for the treatment of small cell lung cancer, wherein said treatment comprises administering a combination therapy of lurbinectedin and atezolizumab to a patient in need thereof. The small cell lung cancer may be ES-SCLC.

In a further aspect there is provided the use of lurbinectedin and atezolizumab in the manufacture of a medicament for the treatment of small cell lung cancer, wherein said treatment comprises administering a combination therapy of lurbinectedin and atezolizumab to a patient in need thereof. The small cell lung cancer may be ES-SCLC.

In a further aspect there is provided lurbinectedin for use in the treatment of small cell lung cancer, wherein said treatment comprises administering a combination therapy of lurbinectedin and atezolizumab to a patient in need thereof. The small cell lung cancer may be extensive stage small cell lung cancer ES-SCLC.

In a further aspect there is provided atezolizumab for use in the treatment of small cell lung cancer, wherein said treatment comprises administering a combination therapy of lurbinectedin and atezolizumab to a patient in need thereof. The small cell lung cancer may be ES-SCLC.

In a further aspect there is provided lurbinectedin and atezolizumab for use in the treatment of small cell lung cancer, wherein said treatment comprises administering a combination therapy of lurbinectedin and atezolizumab to a patient in need thereof. The small cell lung cancer may be ES-SCLC.

In a further aspect there is provided lurbinectedin for use in the treatment of small cell lung cancer, wherein in said treatment lurbinectedin is administered in combination with atezolizumab to a patient in need thereof. The small cell lung cancer may be ES-SCLC.

In a further aspect there is provided atezolizumab for use in the treatment of small cell lung cancer, wherein in said treatment atezolizumab is administered in combination with lurbinectedin to a patient in need thereof. The small cell lung cancer may be ES-SCLC.

In a further aspect there is provided lurbinectedin and atezolizumab for use in the treatment of small cell lung cancer, wherein in said treatment lurbinectedin is administered in combination with atezolizumab to a patient in need thereof. The small cell lung cancer may be ES-SCLC.

Dosage forms, pharmaceutical packages and preparations, and kits of parts are also provided by the invention. These may comprise lurbinectedin and/or atezolizumab packaged for use in a method of treatment of small cell lung cancer, wherein said treatment comprises administering a combination therapy of lurbinectedin and atezolizumab to a patient in need thereof according to the present invention. The dosage forms, packages, preparations and kits may further comprise instructions for providing treatment to a patient according to the present invention.

The following embodiments apply to all aspects of the present invention.

Lurbinectedin and atezolizumab may be administered concurrently, separately or sequentially. Multiple administrations of either lurbinectedin, or atezolizumab, or both, may be given.

Lurbinectedin may be administered at a dose of between 2.5 and 3.2 mg/m². In a preferred embodiment lurbinectedin is administered at a dose of 2.5 mg/m², preferably at a dose of 2.8 mg/m², more preferably at a dose of 3.0 mg/m², even more preferably at a dose of 3.1 mg/m², and most preferably at a dose of 3.2 mg/m². In a preferred embodiment lurbinectedin is administered at a dose between 2.5 to 3.2 mg/m², preferably 2.8 to 3.2 mg/m², more preferably 3.0 to 3.2 mg/m², even more preferably 3.1 to 3.2 mg/m² and most preferably 3.2 mg/m².

Lurbinectedin and atezolizumab may be administered in cycles once every one to four weeks, preferably once every three weeks or once every four weeks. A particular administration cycle is one every 21 days. Day 1 q3 wk.

Any suitable administration route may be used, for example, subcutaneous, intravenous, intraperitoneal. Different administration routes may be used for the lurbinectedin and atezolizumab.

Lurbinectedin may be administered by intravenous infusion. Lurbinectedin may be administered after atezolizumab.

Lurbinectedin may be administered as an infusion, preferably with an infusion time of up to 24 hours, 1 to 12 hours, 1 to 6 hours and most preferably 1 hour. In embodiments, dosing may be −5 minutes to +20 minutes of the stated infusion time.

Lurbinectedin may be administered on day 1. Lurbinectedin may be administered on day 1 of a 21-day cycle. A window of +/−2 days may be allowed for administration on day 1 of the cycle. A window may not be allowed in cycle 1.

Lurbinectedin may be administered in the form of a pharmaceutically acceptable salt selected from the hydrochloride, hydrobromide, hydroiodide, sulfate, nitrate, phosphate, acetate, trifluoroacetate, maleate, fumarate, citrate, oxalate, succinate, tartrate, malate, mandelate, methanesulfonate, p-toluenesulfonate, sodium, potassium, calcium and ammonium salts, ethylenediamine, ethanolamine, N,N-dialkylenethanolamine, triethanolamine and basic amino acids salts.

Atezolizumab may be administered at a dose of 840 mg to 1680 mg, preferably 900 mg to 1500 mg, 1000 mg to 1400 mg, 1100 mg to 1300 mg. In a preferred embodiment, atezolizumab is administered at a dose 1200 mg. In an embodiment, atezolizumab may be administered as 840 mg every two weeks, or 1200 mg every three weeks, or 1680 mg every four weeks. Particularly preferred is 1200 mg every three weeks.

Atezolizumab may be administered by intravenous infusion. Atezolizumab may be administered before lurbinectedin.

Atezolizumab may be administered as an infusion, preferably with an infusion time of up to 24 hours, 1 to 12 hours, 1 to 6 hours and most preferably 60 minutes or 30 minutes for the second and subsequent infusions. In embodiments, dosing may be −5 to +30 minutes of the stated infusion time.

Atezolizumab may be administered on day 1. Atezolizumab may be administered on day 1 of a 21-day cycle. A window of +/−2 days may be allowed for administration on day 1 of the cycle. A window may not be allowed in cycle 1.

In embodiments, the patient may also receive granulocyte-colony stimulating factor G-CSF. In embodiments, in Cycle 1, patients may receive primary prophylaxis with G-CSF starting 24-72 hours after Day 1 of Cycle 1, and for five days. Said another way, the patients may receive G-CSF daily for five days with the first dose starting 24-72 hours after Day 1. In a further embodiment, primary G-CSF prophylaxis for further cycles is administered at the same regimen. In a further embodiment, primary G-CSF prophylaxis for further cycles is administered at the same regimen.

Any number of cycles may be used in accordance with the present invention. In an embodiment, the treatment comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more cycles.

The present invention has identified advantageous dosage regimens useful in the treatment of cancer.

The cancer may be a solid tumor.

The solid tumour may be selected from the group consisting of prostate cancer, breast cancer, lung cancer, colorectal cancer, melanomas, bladder cancer, brain/CNS cancer, cervical cancer, esophageal cancer, gastric cancer, head/neck cancer, kidney cancer, liver cancer, lymphomas, ovarian cancer, pancreatic cancer, and sarcomas.

The lung cancer may be selected from mesothelioma, malignant mesothelioma, malignant pleural mesothelioma, malignant peritoneal mesothelioma, preferably malignant pleural mesothelioma.

The lung cancer may be non-small cell lung cancer.

The lung cancer may be small cell lung cancer.

The small cell lung cancer may be extensive stage small cell lung cancer (ES-SCLC).

The patient may have progressed, including wherein the patient has progressed from first line therapy. The patient may be pre-treated. The patient may be heavily pre-treated. The patient may be progressive.

The cancer may be advanced.

The patient may have progressed from prior platinum treatment.

The patient may be immunotherapy naive.

The treatment may be second line treatment.

The treatment may result in one or more of the following outcomes: reduction in tumour size; delay in growth of tumour; prolongation of life of the patient; remission. These outcomes may be in comparison to a control subject (or hypothetical control subject) not given the treatment, or given an alternative treatment.

The patient may have progressed from prior immunotherapy.

The patient may have progressed from standard of care. The patient may have progressed from standard of care which includes immunotherapy.

The patient may have progressed from platinum-etoposide, e.g. cisplatin-etoposide or carboplatin-etoposide; carboplatin-oral topotecan; cisplatin-irinotecan; or carboplatin-gemcitabine.

The patient may have progressed from an immune checkpoint inhibitor together with a platinum agent and etoposide. Examples include (but are not limited to) carboplatin-etoposide-atezolizumab, or platinum-etoposide-durvalumab.

The patient may have progressed from nivolumab-ipilimumab.

### BRIEF DESCRIPTION OF THE FIGURES

FIG. 1 shows Progression Free Survival (PFS) results in advanced small cell lung cancer that progressed following prior therapy with platinum-based chemotherapy.

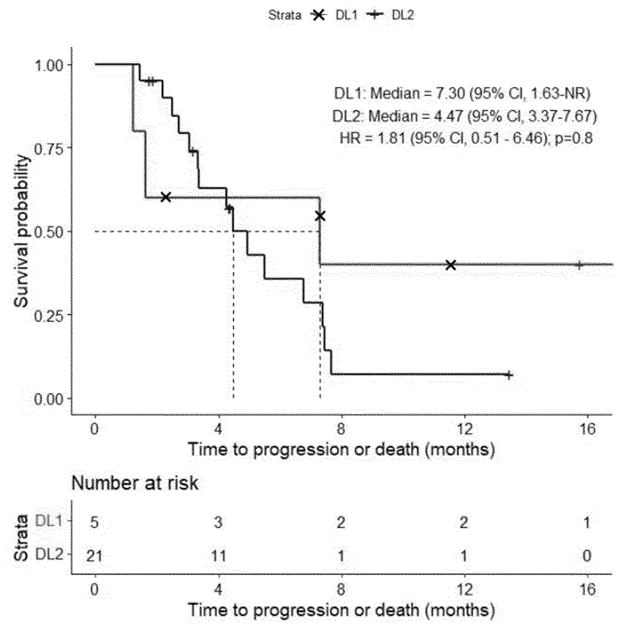

### DETAILED DESCRIPTION OF THE INVENTION

In the present application, a number of general terms and phrases are used, which should be interpreted as follows.

The term “treating” as used herein, unless otherwise indicated, means reversing, attenuating, alleviating or inhibiting the progress of the disease or condition to which such term applies, or one or more symptoms of such disorder or condition. The term “treatment” as used herein, unless otherwise indicated, refers to the act of treating as “treating” is defined immediately above.

“Patient” includes humans, non-human mammals (e.g. dogs, cats, rabbits, cattle, horses, sheep, goats, swine, deer, and the like) and non-mammals (e.g. birds, and the like).

“Lurbinectedin” (PM01183) is a new synthetic tetrahydroisoquinoline alkaloid that which binds the DNA minor groove causing spatial distortion of DNA and protein complexes and leading to the formation of DNA double-strand breaks (DSBs), thus inducing apoptosis and delaying progression through the cell cycle S/G2 phase. Lurbinectedin has the following structure:

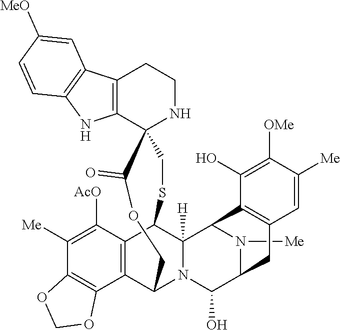 [SMILES: COc1ccc2[nH]c3c(c2c1)CCN[C@]31CS[C@@H]2c3c(OC(C)=O)c(C)c4c(c3[C@H](COC1=O)N1[C@@H]2[C@H]2c3c(cc(C)c(OC)c3O)C[C@@H]([C@@H]1O)N2C)OCO4]

In vitro, lurbinectedin demonstrated cytotoxic effects against a broad selection of tumour-derived cell lines with half maximal inhibitory concentration (IC₅₀) values in the low to very low nanomolar range (approximately median IC₅₀ of 1E-10 M). Lurbinectedin also exhibits in vivo antitumour activity against different murine models of xenografted human-derived tumour types.

The antineoplastic in vitro activity of lurbinectedin was evaluated in a panel of solid tumour cell lines (some of which are shown in Table 1), which were exposed to a range of lurbinectedin concentrations for 72 hours and then assayed for viability by a MTT short-term assay.

TABLE 1
Selected in vitro activity of PM01183.

| Tumour | Cell line | IG₅₀ (M) |
|---|---|---|
| Breast | BT-474 | 1.3 · 10⁻⁹ |
| | MDA-MB-231 | 3.5 · 10⁻⁹ |
| | MCF-7 | 1.7 · 10⁻⁹ |
| Colon | LoVo | 2.0 · 10⁻⁹ |
| | HCT 116 | 6.5 · 10⁻⁸ |
| | HT-29 | 2.4 · 10⁻⁹ |
| Lung | A-549 | 1.3 · 10⁻⁹ |
| | NCl-H460 | 1.6 · 10⁻⁹ |
| | NCl-H23 | 5.4 · 10⁻¹⁰ |
| Ovarian | A2780 | 1.6 · 10⁻⁹ |
| | IGROV-1 | 9.8 · 10⁻⁹ |
| Pancreas | MiaPaca-2 | 1.1 · 10⁻⁹ |
| | PANC-1 | 2.9 · 10⁻⁹ |

IG₅₀, concentration that results in 50% of cell growth inhibition.

The antineoplastic in vivo activity of lurbinectedin was demonstrated in a panel of several different human-derived tumour types, i.e., breast, colon, lung, ovarian and prostate (Table 2). The resulting tumour susceptibility was analyzed in xenografts grown in athymic mice, when unformulated lurbinectedin was administered at the rodent maximum tolerated dose [0.3 mg/kg (0.9 mg/in)] as single bolus intravenous (i.v.) injection. Lurbinectedin demonstrated statistically significant antitumour activity (p<0.05) against breast, lung and ovarian xenografts at different time points during the experiment, but had a more moderate antitumour profile against bladder, pancreas and prostate.

TABLE 2
Selected in vivo activity of PM01183.

| | | | Dose level | | |
|---|---|---|---|---|---|
| | Cell | | mg/kg/day | T/C | Optimal |
| Tumour | line | Schedule | (mg/m²/day) | % | day |
| Lung | LXFL 529 | Q7d × 3 | 0.18 (0.54) | 5 | D-28 |
| Bladder | UM-UC-3 | Qd × 5 × 2 | 0.06 (0.18) | 58 | D-23 |
| Breast | MDA-MB-231 | Q7d × 3 | 0.18 (0.54) | 40 | D-34 |
| | MX-1 | Q7d × 3 | 0.18 (0.54) | 0 | D-21 |
| Ovary | A2780 | Q7d × 3 | 0.18 (0.54) | 34 | D-17 |
| Pancreas | Capan-1 | Q7d × 3 | 0.18 (0.54) | 61 | D-61 |
| Prostate | PC-3 | Q7d × 3 | 0.18 (0.54) | 65 | D-27 |

D, day; Qd × 5 × 2, two cycles of five daily doses; Q7d × 3, three consecutive weekly doses (D-0, 7, 14); T/C, treatment/control.

Toxicology studies in rats and dogs showed that the main target organs were the bone marrow and the liver. The effect of a single bolus injection of PM01183 on cardiovascular parameters [arterial blood pressure (ABP), heart rate (HR) and lead II electrocardiogram (ECG)] was evaluated in dogs for six hours. This study showed no effects on heart, ABP, lead II ECG variables (PR, QT, QTcF and QTcV intervals, and QRS duration), ECG gross morphology or rhythm in dogs treated with PM01183 at doses up to 0.01 mg/kg (0.2 mg/m²). Additionally, two different toxicity studies found no electrophysiological alterations in the HR and the ECGs of dogs following single or repeated lurbinectedin administration at doses up to 0.05 mg/kg (1 mg/m²).

### Clinical Data

Based on the positive preclinical results described above, the clinical development program of lurbinectedin was started in March 2009. Currently, this program comprises four phase I single-agent studies (three in solid tumours and one in acute leukemia); six phase Ib combination studies (with gemcitabine, capecitabine, doxorubicin, cisplatin, irinotecan, or paclitaxel with or without bevacizumab in selected advanced solid tumours); five phase II studies (four with lurbinectedin as single agent in second-line pancreatic cancer, in BRCA-mutated or in BRCA-unselected metastatic breast cancer patients and in platinum-resistant/refractory ovarian cancer, and also selected advanced solid tumours; and one in combination with gemcitabine as second-line therapy in advanced non-small cell lung cancer [NSCLC]); two phase III studies (one comparing single-agent lurbinectedin vs. pegylated liposomal doxorubicin [PLD] or topotecan in platinum-resistant ovarian cancer, and one comparing lurbinectedin in combination with doxorubicin vs. cyclophosphamide, doxorubicin and vincristine [CAV] or topotecan in small cell lung cancer [SCLC]); one QT evaluation study in patients with normal cardiac conduction and function, systolic blood pressure of 90-150 mmHg and normal serum electrolyte levels already participating in the phase II trial PM1183-B-005-14; and two investigator-sponsored studies (ISTs: one with PM01183 in combination with olaparib in advanced solid tumours; and one with lurbinectedin alone or in combination with doxorubicin or gemcitabine in soft tissue sarcoma). As of 15 Jan. 2017 (cutoff date for IB version 9.0), 1515 patients have been enrolled in lurbinectedin clinical studies and 1204 have been treated with lurbinectedin-containing therapy: 460 in phase I trials, 448 in phase II trials, 234 in phase III trials and 62 in ISTs.

The completed PM1183-A-001-08 phase I trial evaluated i.v. lurbinectedin in human patients for the first time, when infused over one hour (h) every three weeks (q3 wk) in 31 patients with advanced and refractory solid tumours. Among these, 15 (48.4%) patients were treated at the defined recommended dose (RD). The RD for phase II studies was defined at a PM01183 dose of 4.0 mg/m² q3 wk-equivalent to a 7.0 mg flat dose (FD) q3 wk. Treatment at the RD was generally well tolerated with standard antiemetic prophylaxis. The most relevant toxicity at the RD was reversible, short-lasting myelosuppression. One patient had a grade 4 thrombocytopenia, the only dose-limiting toxicity (DLT) at the RD. No signs of cumulative toxicity were observed. Antitumour activity was observed at the RD. The pharmacokinetic (PK) analysis showed high inter-patient variability (>50.0% in the area under the curve [AUC]), and linearity across all explored doses. Linear regression analysis showed correlation between neutropenia and AUC (r²=0.452).

Two other phase I trials have been completed: one with single-agent lurbinectedin with a Day −1 and −8 q3 wk schedule in solid tumours (PM1183-A-005-11) and one with lurbinectedin in combination with gemcitabine in selected advanced solid tumours (PM1183-A-004-10). Antitumour activity has been observed in both studies.

In addition, recruitment has been closed in five phase I trials: one with single-agent lurbinectedin in acute relapsed/refractory adult leukemia and myelodysplastic syndrome (PM1183-A-002-10), and four with lurbinectedin in combination with doxorubicin (PM1183-A-003-10), capecitabine (PM1183-A-006-12), paclitaxel with or without bevacizumab (PM1183-A-007-13), and cisplatin (PM1183-A-008-13) in selected solid tumours. Recruitment is currently ongoing in two phase I trials: one with single-agent lurbinectedin in Japanese patients with unresectable/advanced solid tumours (PM1183-A-013-15) and the present study with lurbinectedin in combination with irinotecan in selected advanced solid tumours (PM1183-A-014-15).

Of the five phase II trials, two that evaluated lurbinectedin as second-line treatment in advanced pancreatic cancer (PM1183-B-001-10) and in platinum-resistant and platinum-refractory ovarian cancer (PM1 183-B-002-11) have been completed. The other three phase II trials are ongoing (although recruitment is closed in one) and are evaluating lurbinectedin as second-line treatment in BRCA 1/2-associated or unselected breast cancer (PM1183-B-003-11), in NSCLC, either alone or in combination with gemcitabine (PM1183-B-004-13), and in several selected advanced solid tumours: SCLC, head and neck carcinoma (H&N), neuroendocrine tumours (NETs), biliary tract carcinoma, endometrial carcinoma, BRCA 1/2-associated metastatic breast carcinoma, carcinoma of unknown primary site, germ cell tumours (GCTs), and Ewing's family of tumours (EFTs) (PM1183-B-005-14).

Both phase III studies are ongoing. Recruitment has been closed in the one comparing single-agent lurbinectedin vs. PLD or topotecan in platinum-resistant ovarian cancer (PM1183-C-004-14 [CORAIL]), and is ongoing in the one comparing lurbinectedin in combination with doxorubicin vs. CAV ortopotecan in SCLC (PM1183-C-003-14 [ATLANTIS]).

Recruitment has also been closed in the QT evaluation study (PM1183-B-005-14-QT) and is ongoing in both ISTs (IST POLA/ACOG1401 in advanced solid tumours, and IST 15-083 in soft tissue sarcoma.

A pooled data logistic regression analysis of phase II data suggested that grade 3/4 neutropenia and thrombocytopenia could be related to body surface area (BSA). Owing to these findings, all actively recruiting and planned studies now use BSA-based dosing to limit severe myelosuppression. The original RD of 4.0 mg/m² q3 wk has been reviewed and reduced to 3.2 mg/m² q3 wk for the same reason.

Further information regarding the mechanism of action and in vivo efficacy of lurbinectedin can be found in 100^{th} AACR Annual Meeting, Apr. 18-22, 2009, Denver, CO, Abstract Nr. 2679 and Abstract Nr. 4525; Leal J F M et. al. Br. J. Pharmacol. 2010, 161, 1099-1110; and Belgiovine, C et al. Br. J. Cancer, 2017; 117(5): 628-638.

Further information regarding the clinical development of PM01183 (lurbinectedin) can be found in:
Elez, M E. et. al. Clin. Cancer Res. 2014, 20(8), 2205-2214;
50^{th} ASCO Annual Meeting, May 30-Jun. 3, 2014, Chicago, IL, Abstract 5505;
26^{th} EORTC—26th EORTC-NCI-AACR Symposium on Molecular Targets and Cancer Therapeutics; Nov. 18-21, 2014, Barcelona, Spain, published in Eur. J. Cancer 2014, 50 (Suppl.6), pages 13-14, Abs. No. 23.
51^{th} ASCO Annual Meeting, May 29-Jun. 2, 2015, Chicago, IL, Abstract No. TPS2604 and Abstract Nr. 7509, published in J. Clin. Oncol. 33, 2015 (suppl);
54^{th} ASCO Annual Meeting, Jun. 1-5, 2018, Chicago, IL, Abstract No. 11519, published in J.
Clin. Oncol. 36, 2018 (suppl);
Cruz, C. et al. J. Clin. Oncol. 2018, 36(31), 3134-3143;
54^{th} ASCO Annual Meeting, Jun. 1-5, 2018, Chicago, IL, Abstract No. 8570, published in J. Clin. Oncol. 36, 2018 (suppl).

Further information may be found in Xie et al Lurbinectedin synergizes with immune checkpoint blockade to generate anticancer; immunity ONCOIMMUNOLOGY, 2019, VOL. 8, NO. 11, e1656502 (9 pages).

The term “lurbinectedin” is intended here to cover any pharmaceutically acceptable salt, ester, solvate, hydrate, prodrug, or any other compound which, upon administration to the patient is capable of providing (directly or indirectly) the compound as described herein. However, it will be appreciated that non-pharmaceutical acceptable salts also fall within the scope of the invention since those may be useful in the preparation of pharmaceutically acceptable salts. The preparation of salts can be carried out by methods known in the art.

For instance, pharmaceutically acceptable salts of the compounds provided herein are synthesized from the parent compounds, which contain a basic or acidic moiety, by conventional chemical methods. Generally, such salts are, for example, prepared by reacting the free acid or base of these compounds with a stoichiometric amount of the appropriate base or acid in water or in an organic solvent or in a mixture of both. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, nitrate, phosphate, and organic acid addition salts such are, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts.

Any compound that is a prodrug of lurbinectedin is within the scope and spirit of the invention. The term “prodrug” is used in its broadest sense and encompasses those derivatives that are converted in vivo to PM01183. The prodrug can hydrolyze, oxidize, or otherwise react under biological conditions to provide PM01183. Examples of prodrugs include, but are not limited to, derivatives and metabolites of PM01183 that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by Burger in “Medicinal Chemistry and Drug Discovery” 6^{th} ed. (Donald J. Abraham ed., 2001, Wiley) and “Design and Application of Prodrugs” (H. Bundgaard ed., 1985, Harwood Academic Publishers).

In addition, any drug referred to herein may be in crystalline or amorphous form either as free compounds or as solvates (e.g. hydrates) and it is intended that all forms are within the scope of the present invention. Methods of solvation are generally known within the art.

Moreover, lurbinectedin for use in accordance with the present invention may be prepared following the synthetic process such as the one disclosed in WO 03/014127, which is incorporated herein by reference.

Atezolizumab (MPDL3280A) is a humanized IgG1 monoclonal antibody consisting of two heavy chains (448 amino acid residues each) and two light chains (214 amino acid residues each) and is produced in Chinese hamster ovary cells. Atezolizumab was engineered to eliminate Fc-effector function via a single amino acid substitution at position 298 on the heavy chain, which results in a non-glycosylated antibody that has minimal binding to Fc receptors and prevents Fc-effector function as expected concentrations in humans.

Atezolizumab targets human DP-L1 and inhibits its interaction with its receptors, programmed cell death protein 1 (PD-1) and B7.1 (CD80, B7-1). Both of these interactions are reported to provide inhibitory signals to T cells.

Atezolizumab is being investigated as a potential therapy against solid tumors and hematologic malignancies in humans.

On 18 Mar. 2016, U.S. FDA and 21 Sep. 2017, EMA approved atezolizumab for the treatment of patients with metastatic NSCLC whose disease progressed during or following platinum-containing chemotherapy. Patients with epidermal growth factor receptor (EGFR) or anaplastic lymphoma kinase (ALK) genomic tumor aberrations should have disease progression on FDA-approved therapy for these aberrations prior to receiving atezolizumab.

This approval was based on two international, randomized, open-label clinical trials (Study G028915 [OAK] and Study G028753 [POPLAR] that demonstrated consistent results in efficacy and safety in a total of 1137 patients with NSCLC.

In 2019, atezolizumab, in combination with carboplatin and etoposide, received approval in Europe for the first-line treatment of adult patients with extensive-stage small cell lung cancer (ES-SCLC)

The combination of the present invention have been found to be useful in the treatment of small cell lung cancer.

“Small cell lung cancer (SCLC)” is a fast growing form of lung cancer. It is sometimes called oat cell cancer. Lung cancer is the leading cause of cancer death in both men and women in the United States. In 1998, an estimated 171,500 new cases were diagnosed, and about 160,100 deaths resulted from this disease. More women die from lung cancer that breast, ovarian and uterine cancer combined, and 4 times as many men die from lung cancer than from prostate cancer.

Lung cancer is a disease in which malignant (cancer) cells form in the tissues of the lung. The two major types of lung cancer are small cell lung cancer (SCLC) and non-small cell lung cancer (NSCLC). SCLC comprises only about 13-15% of all lung cancers at diagnosis; however, SCLC is the more aggressive form or lung cancer. With SCLC, the cancer cells tend to grow quickly and travel to other parts of the body, or metastasize, more easily. Its incidence is associated with smoking, almost two thirds of patients present with advanced disease, and although response rates to chemotherapy are high, the benefit is short-lived. The median survival of patients with untreated SCLC is two to four months. The most common regimens include cisplatin or carboplatin and etoposide. Unfortunately, despite the 40-90% response rate to first-line chemotherapy, long-term survival is unusual because patients develop resistance to chemotherapy and relapse. The overall expected mean survival after disease relapse without treatment was typically two to four months.

However, on Mar. 18, 2019, U.S. FDA approved atezolizumab in combination with carboplatin and etoposide, for the first-line treatment of adult patients with extensive-stage small cell lung cancer (ES-SCLC).

Approval was based on IMpower133 (NCT02763579), a randomized (1:1), multicenter, double-blinded, placebo-controlled trial in 403 patients with ES-SCLC who received no prior chemotherapy for extensive stage disease and had ECOG performance status 0 or 1. Patients were randomized to one of the following:
atezolizumab 1200 mg and carboplatin AUC 5 mg/mL/min on day 1 and etoposide 100 mg/m² intravenously on days 1, 2 and 3 of each 21-day cycle for a maximum of 4 cycles, followed by atezolizumab 1200 mg once every 3 weeks until disease progression or unacceptable toxicity, or
placebo and carboplatin AUC 5 mg/mL/min on day 1 and etoposide 100 mg/m2 intravenously on days 1, 2, and 3 of each 21-day cycle for a maximum of 4 cycles, followed by placebo once every 3 weeks until disease progression or unacceptable toxicity.

Major efficacy outcome measures were overall survival (OS) and progression-free survival (PFS) as assessed by investigator per RECIST 1.1 in the intent-to-treat population. Median OS was 12.3 months (10.8, 15.9) for patients receiving atezolizumab with chemotherapy and 10.3 months (9.3, 11.3) for those receiving placebo with chemotherapy (hazard ratio 0.70; 95% Cl: 0.54, 0.91; p=0.0069). Median PFS was 5.2 months (4.4, 5.6) compared with 4.3 months (4.2, 4.5) in the atezolizumab and placebo arms, respectively (HR 0.77; 0.62, 0.96; p=0.0170).

The most common adverse reaction reported in ≥20% of patients who received atezolizumab in IMpower133 were fatigue/asthenia, nausea, alopecia, constipation and decreased appetite.

In addition, in June 2020, the FDA approved lurbinectedin for the treatment of adult patients with metastatic small cell lung cancer (SCLC) with disease progression on or after platinum-based chemotherapy. Efficacy was demonstrated in the PM1183-B-005-14 trial (Study B-005; NCT02454972), a multicenter open-label, multi-cohort study enrolling 105 patients with metastatic SCLC who had disease progression on or after platinum-based chemotherapy. Patients received lurbinectedin 3.2 mg/m² by intravenous infusion every 21 days until disease progression or unacceptable toxicity. The main efficacy outcome measures were confirmed overall response rate (ORR) determined by investigator assessment using RECIST 1.1 and response duration. Among the 105 patients, the ORR was 35% (95% CI: 26%, 45%), with a median response duration of 5.3 months (95% CO: 4.1, 6.4). The ORR as per independent review committed was 30% (95% CI: 22%, 40%) with a median response duration of 5.1 months (95% CI: 4.9, 6.4). the recommended lurbinectedin dose is 3.2 mg/m² every 21 days.

In embodiments, the cancer is extensive-stage small cell lung cancer (ES-SCLC). In embodiments, the cancer is extensive-stage small cell lung cancer (ES-SCLC) where the cancer has spread beyond a single area that can be treated with radiotherapy. It might have spread within the chest (either widely throughout the lung, to the other lung or to lymph nodes further away from the cancer) or to other parts of the body. Or there may be cancer cells in the fluid around the lung (a malignant pleural effusion).

“Malignant mesothelioma” is a disease in which malignant (cancer) cells are found in the pleura (the thin layer of tissue that lines the chest cavity and covers the lungs) or the peritoneum (the thin layer of tissue that lines the abdomen and covers most of the organs in the abdomen). Malignant mesothelioma may also form in the heart or testicles, but this is rare. The four types of mesothelioma are therefore pleural (lung lining), peritoneal (abdominal lining), pericardial (heart sac) and testicular.

Mesothelioma can also be identified by three cancer cell types: epithelioid, sarcomatoid and biphasic, and can therefore be defined as epithelioid mesothelioma (epithelioid cells), sarcomatoid mesothelioma (sarcomatoid cells) or biphasic mesothelioma (epithelioid and sarcomatoid cells). Pleural is the most common mesothelioma. Approximately 70% to 75% of cases occur in the pleura. Peritoneal disease accounts for 10% to 20% of mesothelioma cases. There is less research available on peritoneal compared to pleural; however, the prognosis for this tumor type is better. Pericardial Mesothelioma is extremely rare. Around 200 cases are reported in medical literature. Testicular mesothelioma develops in the lining of the testes. This form of mesothelioma is the most rare. Less than 100 cases are reported in the medical literature.

The three mesothelioma cell varieties are epithelial, sarcomatoid and biphasic. Biphasic is a mix of the first two cell types. Different mesothelioma tumors respond differently to treatment. Epithelial or epithelioid cells typically respond the best to treatment, and sarcomatoid cells are typically more resistant to treatment. Epithelioid mesothelioma makes up approximately 70% to 75% of all cases of asbestos-related mesothelioma cancers. Epithelioid cell typically has the best prognosis. It tends to be less aggressive and doesn't spread as quickly as sarcomatoid and biphasic cell disease. About 50% of pleural disease is epithelioid. Around 75% of peritoneal tumors are made up of epithelioid cells. Sarcomatoid is the least common mesothelioma cell category. It is typically the most aggressive and difficult to treat. It accounts for around 10% to 20% of all mesothelioma diagnoses. About 20% of pleural tumors are sarcomatoid, while only 1% of peritoneal mesothelioma are sarcomatous. Biphasic mesothelioma refers to tumors that contain epithelial and sarcomatoid cells. Life expectancy after diagnosis with biphasic mesothelioma depends upon which cell predominates in the tumor. More epithelioid cells generally mean a better prognosis. If the tumor is mostly sarcomatous, it is harder to treat and life expectancy is shorter. Around 30% of pleural and 25% of peritoneal tumors are biphasic cell.

### Prevalence of Mesothelioma Tumors by Cell Type

| Cell Type | Pleural | Peritoneal |
|---|---|---|
| Epithelioid | 50% | 75% |
| Biphasic | 30% | 24% |
| Sarcomatoid | 20% | 1% |

Based on the limited number of cases reported in the medical literature, pericardial mesothelioma exhibits roughly equal distribution of the three mesothelioma cell types. Approximately two-thirds of testicular mesothelioma cases are epithelioid cell. The rest of testicular cases are biphasic. Only one case of purely sarcomatoid cell disease is reported for testicular mesothelioma.

In an embodiment, the present invention is useful in the treatment of malignant pleural mesothelioma (MPM).

The malignant mesothelioma to be treated may be epithelioid. The malignant mesothelioma to be treated may be sarcomatoid. The malignant mesothelioma to be treated may be biphasic.

The patient may be immunotherapy naive.

The patient may have progressed from prior immunotherapy. By “progressed from prior immunotherapy” it is meant that the patient has previously received immunotherapy.

The immunotherapy may be an immune checkpoint inhibitor. The immune checkpoint inhibitor may be (but is not limited to) a PD-1 inhibitor, for example pembrolizumab, nivolumab or cemiplimab. The immune checkpoint inhibitor may be (but is not limited to) a PD-L1 inhibitor, for example atezolizumab, avelumab, or durvalumab. The immune checkpoint inhibitor may be (but is not limited to) a CTLA-4 inhibitor, for example ipilimumab. The immune checkpoint inhibitor may be (but is not limited to) a LAG-3 inhibitor, for example relatlimab.

The treatment may be second line treatment.

The patient may have progressed from receiving standard of care. Standard of care for ES-SCLC may be: platinum-etoposide, e.g. cisplatin-etoposide or carboplatin-etoposide; carboplatin-oral topotecan; cisplatin-irinotecan; or carboplatin-gemcitabine.

The patient may have progressed from platinum-etoposide, e.g. cisplatin-etoposide or carboplatin-etoposide; carboplatin-oral topotecan; cisplatin-irinotecan; or carboplatin-gemcitabine.

The patient may have progressed from receiving standard of care which includes immunotherapy. Standard of care for ES-SCLC may be an immune checkpoint inhibitor together with a platinum agent and etoposide. Examples include (but are not limited to) carboplatin-etoposide-atezolizumab, or platinum-etoposide-durvalumab. Standard of care for malignant pleural mesothelioma may be nivolumab-ipilimumab.

The patient may have progressed from an immune checkpoint inhibitor together with a platinum agent and etoposide. Examples include (but are not limited to) carboplatin-etoposide-atezolizumab, or platinum-etoposide-durvalumab.

The patient may have progressed from nivolumab-ipilimumab.

In embodiments, the present invention provides dosing schedules to treat the cancer defined herein.

In a preferred embodiment lurbinectedin is administered at a dose of 2.5 mg/m², preferably at a dose of 2.8 mg/m², more preferably at a dose of 3.0 mg/m², even more preferably at a dose of 3.1 mg/m², and most preferably at a dose of 3.2 mg/m².

In a preferred embodiment lurbinectedin is administered at a dose between 2.5 to 3.2 mg/m², preferably 2.8 to 3.2 mg/m², more preferably 3.0 to 3.2 mg/m², even more preferably 3.1 to 3.2 mg/m² and most preferably 3.2 mg/m².

In a preferred embodiment, atezolizumab may be administered at a dose of 840 mg to 1680 mg, preferably 900 mg to 1500 mg, 1000 mg to 1400 mg, 1100 mg to 1300 mg. In a particularly preferred embodiment, atezolizumab is administered at a dose 1200 mg. In an embodiment, atezolizumab may be administered as 840 mg every two weeks, or 1200 mg every three weeks, or 1680 mg every four weeks. Particularly preferred is 1200 mg every three weeks.

In a particularly preferred embodiment, lurbinectedin is administered at a dose between 2.5 to 3.2 mg/m² and atezolizumab is administered at a dose 1200 mg.

In a particularly preferred embodiment, lurbinectedin is administered at a dose of 3.2 mg/m² and atezolizumab is administered at a dose of 1200 mg.

The patient may also receive prophylactic medication whilst getting treatment as described in the present invention. Prophylactic medication includes corticosteroids and 5-HT₃ receptor antagonists. Particular corticosteroids include dexamethasone. Particular 5-HT₃ receptor antagonists include ondansetron. Particular dosages include dexamethasone 8 mg i.v. (or an equivalent dose of another i.v. corticosteroid) and ondansetron 8 mg i.v. (or an equivalent dose of another i.v. 5-HT₃ receptor antagonist). Prophylactic medication can be administered on Day 1 of each cycle. In addition, further prophylactic medication may be administered as needed. An example includes metoclopramide or equivalent, which in embodiments may be administered every eight hours. After Day 1 and Day 8 of each cycle extended oral corticosteroids (for example dexamethasone not exceeding 20 mg/days) and/or 5-HT₃ receptor antagonists (for example oral (or i.v.) ondansetron 4-8 mg (or equivalent)) may be administered.

The patient may also be administered granulocyte-colony stimulating factor G-CSF such as non-pegylated filgrastim. In embodiments, in cycle 1, patients may receive primary prophylaxis with G-CSF starting 24-72 hours after Day 1 of Cycle 1, and during five days. Primary G-CSF prophylaxis for further cycles may be administered at the same regimen, but could also be administered according to physician discretion.

Pharmaceutical compositions comprising lurbinectedin or a pharmaceutically acceptable salt or ester thereof, and a pharmaceutically acceptable carrier may be formulated according to the chosen route of administration. Examples of the administration form include without limitation oral, topical, parenteral, sublingual, rectal, vaginal, ocular and intranasal. Parenteral administration includes subcutaneous injections, intravenous, intramuscular, intrasternal injection or infusion techniques. Preferably, the compositions are administered parenterally. Pharmaceutical compositions can be formulated so as to allow a compound to be bioavailable upon administration of the composition to an animal, preferably human. Compositions can take the form of one or more dosage units, where for example, a tablet can be a single dosage unit, and a container of a compound may be contain the compound in liquid or in aerosol form and may hold a single or a plurality of dosage units.

The pharmaceutically acceptable carrier or vehicle can be particulate, so that the compositions are, for example, in tablet or powder form. The carrier(s) can be liquid, with the compositions being, for example, an oral syrup or injectable liquid. In addition, the carrier(s) can be gaseous, or liquid so as to provide an aerosol composition useful in, for example, inhalatory administration. Powders may also be used for inhalation dosage forms. The term “carrier” refers to a diluent, adjuvant, or excipient, with which the compound according to the present invention is administered. Such pharmaceutical carriers can be liquids, such as water and oils including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, disaccharides, and the like. In addition, auxiliary, stabilizing, thickening, lubricating and coloring agents can be used. In one embodiment, when administered to an animal, the compounds and compositions and pharmaceutically acceptable carriers are sterile. Water is a preferred carrier when the compounds are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monoestearate, talc, sodium chloride, dried skim milk, glycerol, propylene glycol, water, ethanol and the like. The present compositions, if desired, can also contain minor amounts of wetting or emulsifying agents, or pH buffering agents.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide, sweetening agent such as sucrose or saccharin; a flavoring agent such as peppermint, methyl salicylate or orange flavoring; and coloring agent.

When the composition is in the form of a capsule (e.g. a gelatin capsule), it can contain, in addition to materials of the above type, a liquid carrier such as polyethylene glycol, cyclodextrins or a fatty oil.

The composition can be in the form of a liquid, e.g. an elixir, syrup, solution, emulsion or suspension. The liquid can be useful for oral administration or for delivery by injection. When intended for oral administration, a composition can comprise one or more of a sweetening agent, preservatives, dye/colorant and flavour enhancer. In a composition for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intracerebral, intraventricular, intrathecal, intravaginal or transdermal. The preferred mode of administration is left to the discretion of the practitioner, and will depend in part upon the site of the medical condition. In a more preferred embodiment, the compound(s) according to the present invention are administered intravenously. Infusion times of up to 24 hours are preferred to be used, more preferably 1 to 12 hours, with 1 to 6 hours even more preferred and 1 hour most preferred. Short infusion times that allow treatment to be carried out without an overnight stay in a hospital are especially desirable. However, infusion may be 12 to 24 hours or even longer if required. Infusion may be carried out at suitable intervals of, for example, 1 to 4 weeks, and preferably once every three weeks.

In a preferred embodiment atezolizumab is administered at a fixed dose of 1200 mg intravenously (i.v.) as a 60 min infusion (the second and subsequent infusions may be administered over 30 min), followed by lurbinectedin at a dose of 3.2 mg/m² i.v. as a 1-hour infusion on Day 1 every three weeks (q3 wk). A cycle is defined as an interval of three weeks.

Liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides, polyethylene glycols, glycerin or other solvents; antibacterial agents such as benzyl alcohol or methyl paraben; and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant.

The compositions comprise an effective amount of lurbinectedin and/or atezolizumab such that a suitable dosage will be obtained. Administration can be carried out continuously or periodically within the maximum tolerated dose.

In specific embodiments, it can be desirable to administer lurbinectedin or atezolizumab locally to the area in need of treatment. In one embodiment, administration can be by direct injection at the site (or former site) of a cancer, tumour or neoplastic or preneoplastic tissue.

Pulmonary administration can also be employed, e.g. by use of an inhaler or nebulizer, and formulation with an aerosolizing agent, or via perfusion in a fluorocarbon or synthetic pulmonary surfactant. In certain embodiments, lurbinectedin can be formulated as a suppository, with traditional binders and carriers such as triglycerides.

The present composition can take the form of solutions, suspensions, emulsions, tables, pills, pellets, capsules, capsules containing liquids, powders, sustained-release formulations, suppositories, emulsions, aerosols, sprays, suspensions, or any other form suitable for use. Other example of suitable pharmaceutical carriers are described in “Remington's Pharmaceutical Sciences” by E. W. Martin.

The pharmaceutical compositions can be prepared using methodology well known in the pharmaceutical art. For example a composition intended to be administered by injection can be prepared by combining lurbinectedin with water, or other physiologically suitable diluent, such as phosphate buffered saline, so as to form a solution. A surfactant can be added to facilitate the formation of a homogeneous solution or suspension.

Preferred compositions comprising lurbinectedin may invention include:
Pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.
Lyophilised pharmaceutical compositions comprising lurbinectedin and a disaccharide. Particularly preferred disaccharides are selected from lactose, trehalose, sucrose, maltose, isomaltose, cellobiose, isosaccharose, isotrehalose, turanose, melibiose, gentiobiose, and mixtures thereof.

The ratio of lurbinectedin to the disaccharide in embodiments of the present invention is determined according to the solubility of the disaccharide and, when the formulation is freeze dried, also according to the freeze-dryability of the disaccharide. It is envisaged that this lurbinectedin:disaccharide ratio (w/w) can be about 1:10 in some embodiments, about 1:20 in other embodiments, about 1:50 is still further embodiments. It is envisaged that other embodiments have such ratios in the range from about 1:5 to about 1:500, and still further embodiments have such ratios in the range from about 1:10 to about 1:500.

The composition comprising lurbinectedin may be lyophilized. The composition comprising lurbinectedin is usually presented in a vial which contains a specified amount of such compound.

Lurbinectedin may be a lyophilized powder for concentrate for solution for infusion, as 4 mg/vial. Before use, the 4-mg vial may be reconstituted with 8 mL of sterile water for injection, to give a solution containing 0.5 mg/mL of lurbinectedin. For administration to patients as i.v. infusion, reconstituted vials may be diluted with glucose 50 mg/mL (5%) or sodium chloride 9 mg/mL (0.9%) solution for infusion.

The full composition of the PM01183 4 mg vial and the reconstituted solution per mL may be as follows

| | | Concentration per vial |
|---|---|---|
| Component | Concentration/vial | after reconstitution |
| PM01183 | 4.0 mg | 0.5 mg/mL |
| Sucrose | 800 mg | 100 mg/mL |
| Lactic acid | 22.08 mg | 2.76 mg/mL |
| Sodium hydroxide | 5.12 mg | 0.64 mg/mL |

To provide a more concise description, some of the quantitative expressions given herein are not quantified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The invention will now be described further with reference to the following example

### Example

Phase I-II Study to Assess the Safety, Tolerability and Efficacy of PM01183 and atezolizumab in patients with Advanced Small Cell Lung Cancer that Progressed Following Prior Therapy with Platinum-Based Chemotherapy.

### Study Objectives

### Phase I

### Primary Objective:

To determine the maximum tolerated dose (MTD) and the recommended dose for phase II studies (RD) of PM01183 in combination with atezolizumab in advanced SCLC patients progressing after platinum doublet chemotherapy.

### Secondary Objectives:

To characterize the safety profile and feasibility of this combination.
To characterize the pharmacokinetics (PK) of PM01183 and to detect major drug-drug PK interactions.
To obtain preliminary information on the clinical antitumor activity of this combination.
In the event of DLTs being exclusively related to neutropenia, to determine the MTD and the RD of PM01183 in combination with atezolizumab in patients with advanced SCLC with compulsory primary G-CSF prophylaxis.
To evaluate pharmacogenetics (PGt) in germline DNA to identify factors that may explain individual variability in main PK parameters.
To evaluate pharmacogenomics (PGx) in tumor and blood samples to identify potential markers of response and/or resistance.

### Study Design

Prospective, open-label, uncontrolled and multicenter phase I-II study in SCLC patients with ECOG PS 0-1 who have failed one prior platinum-containing line but no more than one chemotherapy-containing line (re-challenge is not allowed). The study will be divided into two parts: a dose-ranging phase I with escalating doses of PM01183 in combination with a fixed dose of atezolizumab, followed by a single-arm phase II part with expansion at the RD determined during the phase 1.

### Phase 1

Patients will receive atezolizumab at a fixed dose of 1200 mg intravenously (i.v.) as a 60-minute infusion (the second and subsequent infusions may be administered over 30 minutes) followed by PM01183 at a starting dose of 2.5 mg/m² i.v. as a 1-hour infusion on Day 1 every three weeks (q3 wk). A cycle is defined as an interval of three weeks. PM01183 doses will be escalated in successive cohorts of patients following a modified Fibonacci scheme and a classical 3+3 design, and according to observed tolerance and safety.

### Study Population

### Inclusion Criteria

1) Voluntarily signed and dated written informed consent prior to any specific study procedure.
2) Age >18 years.
3) Histologically or cytologically confirmed diagnosis of extensive or limited SCLC.
4) Progression to first-line platinum-based chemotherapy.
5) Available tumor tissue blocks or slides from a previous surgery or biopsy.
6) Eastern Cooperative Oncology Group (ECOG) performance status (PS) score ≤1.
7) Measurable disease according to RECIST v. 1.1. Note: irradiated lesions may qualify as target if progression has been documented.
8) At least three weeks since last prior anticancer treatment (including radiotherapy) and recovery to grade ≤1 from any adverse event (AE) related to previous anticancer treatment (excluding sensory neuropathy, anemia, asthenia and alopecia, all grade ≤2) according to the National Cancer Institute Common Terminology Criteria for Adverse Events (NCI-CTCAE, v. 5).
9) Adequate bone marrow, renal, hepatic, and metabolic function (assessed ≤7 days before inclusion in the study):
   a) Platelet count ≥100×109/L, hemoglobin ≥9.0 g/dL and absolute neutrophil count (ANC) ≥1.5×109/L.
   b) Aspartate aminotransferase (AST) and alanine aminotransferase (ALT) 53.0×the upper limit of normal (ULN), independently of the presence of liver metastases.
   c) Alkaline phosphatase (AP) ≤2.5×ULN.
   d) Total bilirubin ≤1.5×ULN or direct bilirubin ≤ULN.
   e) International Normalized Ratio (INR)<1.5 (except if patient is on oral anticoagulation therapy).
   f) Calculated creatinine clearance (CrCL) ≥30 mL/minute (using Cockcroft and Gault's formula).
   g) Creatine phosphokinase (CPK) ≤2.5×ULN.
   h) Albumin ≥3.0 g/dL. Albumin infusion to fulfill the inclusion criterion is forbidden.
   i) Thyroid stimulating hormone (TSH) within institutional normal limits. If TSH is above the ULN, then a free T4 within institutional normal limits is acceptable.
10) Evidence of non-childbearing status for women of childbearing potential (WOCBP). Both women and men must agree to use a highly effective contraceptive measure during the trial, for at least five months after last atezolizumab dose, and for at least six weeks (women) or 4 months (men) after last PM01183 dose. Fertile male patients with WOCBP partners must agree to refrain from fathering a child or donating sperm during the trial and up to five months after treatment discontinuation. Acceptable methods of contraception include abstinence, intrauterine device (IUD), oral contraceptive, subdermal implant and/or double barrier.
   Exclusion criteria
1) Active or untreated central nervous system (CNS) involvement. Treated CNS metastases have to show radiographic stability (defined as no CNS progression for at least three weeks from post-radiotherapy brain scan to brain scan performed prior study entry), and patients should not have neurologic sign/symptoms secondary to the brain metastases or RT. Any steroid treatment must be completed
≥14 days before first dose of study treatment.
2) More than one prior chemotherapy-containing line (re-challenge with the same initial regimen is not allowed).
3) Patients with radiation therapy (RT) in more than 35% of the bone marrow.
4) History of previous bone marrow and/or stem cell transplantation.
5) Impending need for RT (e.g., painful bone metastasis and/or risk of spinal cord compression).
6) History of allergy or hypersensitivity to any of the study drugs or their excipients. 7) Prior therapy with PM01183, antibodies against PD-1, PD-L1, PD-L2, CD137, or cytotoxic T lymphocyte associated antigen-4 (CTLA-4).
8) Live vaccines within 30 days prior to start of study treatment and while on treatment.
9) History of other prior malignancy, with the exception of basal cell carcinoma of the skin, superficial bladder cancer, squamous cell carcinoma of the skin, in situ cervical cancer. Patients with other prior malignancies and no disease recurrence for 3 years are eligible.
10) Concomitant diseases/conditions:
   a) History or presence of unstable angina, myocardial infarction, congestive heart failure defined as abnormal left ventricular ejection fraction (LVEF)<50% assessed by multiple-gated acquisition scan (MUGA) or equivalent by ultrasound (US), or clinically significant valvular heart disease within 12 months prior first study dose.
   b) Symptomatic arrhythmia or any uncontrolled arrhythmia requiring ongoing treatment.
   c) Ongoing chronic alcohol consumption, or cirrhosis with Child-Pugh score B or C.
   d) Active uncontrolled infection. Serious non-healing wound, ulcer or bone fracture.
   e) Diagnose of immunodeficiency or receiving systemic steroids therapy (more than a daily dose of 10 mg of prednisone or equivalent per day) or any other form of immunosuppressive therapy within 14 days prior to the first study dose.
   f) Active autoimmune disease that required systemic treatment in the past two years (i.e., with disease-modifying agents, corticosteroids and immunosuppressive drugs).
   Patients with vitiligo or resolved childhood asthma/atopy are eligible, as well as patients who require intermittent use of bronchodilators or local steroid injections, patients with hypothyroidism stable on hormone replacement, patients with insulin-treated controlled type 1 diabetes or Sjogren's syndrome.
   g) History of idiopathic pulmonary fibrosis, organizing pneumonia, drug-induced pneumonitis, idiopathic pneumonitis or evidence of active pneumonitis on screening chest computed tomography (CT) scans. A history of radiation pneumonitis in radiation field (fibrosis) will be allowed if asymptomatic and not requiring steroids.
h) Known history of active tuberculosis (Mycobacterium tuberculosis).
i) Ongoing treatment-requiring, non-neoplastic chronic liver disease of any origin. For hepatitis B, this includes positive tests for both Hepatitis B surface antigen (HBsAg) and quantitative Hepatitis B polymerase chain reaction (PCR). For hepatitis C, this includes positive tests for both Hepatitis C antibody and quantitative Hepatitis C PCR. Patients taking hepatitis-related antiviral therapy within 6 months prior to the first study dose will also be excluded.
   j) Known human immunodeficiency virus (HIV) infection.
   k) Myopathy or any clinical situation that causes significant and persistent elevation of CPK (>2.5×ULN in two different determinations performed one week apart).
   l) Limitation of the patient's ability to comply with the treatment or follow-up procedures.
   m) Any other major illness that, in the Investigator's judgment, will substantially increase the risk associated with the patient's participation in this study.

### Study Population

### Expected Number of Patients

The number of patients in the phase I part may vary depending both on the tolerability to PM01183 combined with atezolizumab and the number of dose levels required to identify the MTD, estimated approximately 24 patients in phase I.

### Study Drug

### Formulation

### PM01183

PM01183 drug product is provided as a lyophilized powder for concentrate for solution for infusion in 4-mg vials.

Before use, the 4-mg vial should be reconstituted with 8 mL of sterile water for injection to give a solution containing 0.5 mg/mL of PM01183. For administration to patients as an i.v. infusion, reconstituted vials should be diluted either with glucose 50 mg/mL (5%) solution or sodium chloride 9 mg/mL (0.9%) solution for infusion.

The full composition of the PM01183 4-mg vials and the reconstituted solution per mL is shown in Table S1.

TABLE S1
Composition of PM01183 vials.

| | PM01183 | Concentration per vial |
|---|---|---|
| Component | 4 mg | after reconstitution |
| PM01183 | 4.0 mg | 0.05 mg/mL |
| Sucrose | 800 mg | 100 mg/mL |
| Lactic acid | 22.08 mg | 2.76 mg/mL |
| Sodium hydroxide | 5.12 mg | 0.64 mg/mL |

### Atezolizumb

Commercially available presentations of vials containing atezolizumab (1200 mg/20 mL per vial) will be provided as appropriate.

### Treatment Schedule

Atezolizumab at a fixed dose of 1200 mg administered as a 60-minute i.v. infusion (second and subsequent infusions over 30 minutes) followed by PM01183 as a 1-hour i.v. infusion, both on Day 1 q3 wk.

### Administration Route and Dose

### Phase I

Patients will consecutively receive the following on Day 1 q3 wk (three weeks=one treatment cycle):
Atezolizumab: i.v. infusion over 60 minutes (or over 30 minutes for the second and subsequent infusions), immediately followed by:
PM01183: i.v. infusion over one hour at a starting dose of 2.5 mg/m², over a minimum of 100 mL dilution on 5% glucose or 0.9% sodium chloride (at a fixed rate) via a central line (or a minimum of 250 mL dilution if a peripheral line will be used) through a pump device.

During the Phase I part, body surface area (BSA) will be calculated every cycle according to the DuBois formula. PM01183 dose will be recalculated before a new cycle is started. Doses will be rounded to the first decimal.

### Dose Escalation Scheme (Phase I)

The dose escalation scheme will follow pre-defined dose levels, starting at DL1, as summarized in the following table:

TABLE S2
Dose escalation scheme.

| | No. of | Atezolizumab | PM01183 |
|---|---|---|---|
| Dose level | patients | dose (mg) | dose (mg/m²) |
| DL-1^{a} | 0-6 | 1200 | 2.0 |
| DL-1 (starting | 3-6 | 1200 | 2.5 |
| dose) | | | |
| DL2 | 3-6 | 1200 | 3.2 |
| DL3 | 3-9 | 1200 | 3.5 |

^{a}DL-1 will be initiated if DL1 is defined as maximal-tolerated dose.
DL, dose level.

Patients are evaluated for DLTs (dose limiting toxicities) during the complete first treatment cycle (3-week period). Patients not fully evaluable will be replaced.
Cohorts of at least three fully evaluable patients will be initially included at each dose level.
   If the first patient of a dose level has no DLT (within the 3-week period), the second and third patients may be included simultaneously.
   If the first patient has a DLT, the third patient of the dose level will be included only if the second patient has no DLT.
   If one of these three first evaluable patients experiences a DLT during Cycle 1, three more evaluable patients will be included at that dose level.
If no DLT occurs in more than one third of evaluable patients at a dose level, escalation will proceed to the next dose level.
If more than one third of evaluable patients at a given dose experience a DLT, that dose will be considered the MTD (i.e., lowest dose level explored during dose escalation at which more than one third of evaluable patients experience a DLT). Dose escalation will be terminated, except if all DLTs occurring at a given dose level are related to neutropenia (i.e., febrile neutropenia, grade 4 neutropenia lasting >3 days, grade 3 neutropenia lasting >7 days or neutropenic sepsis), in which case dose escalation may be resumed at the same dose level, but with compulsory primary G-CSF prophylaxis.
The dose level immediately below the MTD (or DL3 if the MTD has not been reached) will be expanded in order to have at least nine evaluable patients treated at that dose level (i.e., including escalation and expansion). This level will be confirmed as the RD if less than one third of the first nine evaluable patients experience a DLT (Table S3).

TABLE S3
Dose escalation/expansion scheme.

| No. of patients | No. of patients with | |
|---|---|---|
| evaluable^{a} for DLT | DLTs in Cycle 1 | Action |
| 3 | 0 | Escalate DL |
| | 1 | Add three patients |
| | >1 | MTD |
| 6 | 1 | Escalate DL |
| | >1 | MTD |

^{a}Patients not evaluable for DLT during dose escalation must be replaced.
DL, dose level; DLT, dose-limiting toxicities; MTD, maximum tolerated dose.

According to the toxicity observed, intermediate dose levels may be explored if considered appropriate.
Intra-patient dose escalation will not be allowed under any circumstances.
For DLTs, tables of frequencies were used to characterize the profiles of DLTs. First, all AEs and SAEs were coded according to the 23.0 version of the Medical Dictionary for Regulatory Activities (MedDRA) and their Preferred Term (PT). Then, the frequency tables were obtained and they were displayed by dose level and the use of primary G-CSFG prophylaxis.

### Prophylactic Medication

On Day 1 of each cycle, all patients must receive the following prophylactic medication before infusion of any study drug:
Dexamethasone 8 mg i.v. or equivalent,
Ondansetron 8 mg i.v. or equivalent, with or without metoclopramide 10 mg i.v. or equivalent.

Other possible prophylactic medications:
Extended oral prednisone not exceeding 10 mg/day and/or oral ondansetron 4 to 8 mg or equivalent, at the Investigator's criteria if required.
Additional antiemetics might be used, if required.
Aprepitant and equivalent agents (e.g., fosaprepitant) are forbidden in patients treated with PM01183.

If primary G-CSF prophylaxis is required in specific cohorts of patients, it will consist of G-CSF (non-pegylated filgrastim) 300 μg/day subcutaneously for five consecutive days, starting at least 24 hours after treatment infusion.

### Allowed Medications/Therapies

Therapies for preexisting and treatment-emergent medical conditions, including pain management.
Blood products and transfusions, as clinically indicated.
Bisphosphonates.
In case of nausea or vomiting, secondary prophylaxis and/or symptomatic treatment for emesis according to American Society of Clinical Oncology (ASCO) guidelines (taking into account the aforementioned limit of corticosteroids per day).
Erythropoietin use according to ASCO guidelines.
Secondary prophylaxis or therapeutic use of G-CSF. Note: a patient who develops severe non-febrile neutropenia during Cycle 1 of the phase I study part should not receive therapeutic G-CSF unless the DLT criterion for neutropenia was met or it was clinically indicated.
Palliative limited field bone RT (e.g. for pain control) if needed once Cycle 1 has been completed.
Megestrol acetate for appetite stimulation.

### Prohibited Medications/Therapies

Concomitant administration of any other antineoplastic therapy.
Other investigational agents.
Aprepitant or directly related substances (e.g., fosaprepitant).
Corticosteroids at a dose of >10 mg/day of prednisone or equivalent (excluding premedication for chemotherapy and i.v. contrast).
Immunosuppressive therapies other than corticosteroids.
Primary prophylaxis and/or treatment with colony-stimulating factors (G-CSF) during Cycle 1, (except if the patient[s] is/are treated within an specific cohort resuming dose escalation with primary G-CSF prophylaxis, in which case use is not only allowed but mandatory). Secondary prophylaxis is allowed, if required, during the following cycles, instead of a dose reduction due to exclusively febrile neutropenia or grade 4 neutropenia or upon the Sponsor's agreement.

### Drug-Drug Interactions

In vitro studies with human microsomes showed that CYP3A4 is the major CYP isoform involved in the metabolism of PM01183, followed by CYP2E1, CYP2D6 and CYP2C9. The estimated contribution of the other CYP isoenzymes to the PM01183 metabolism is considered to be negligible. Therefore, concomitant drugs which induce or inhibit any of these cytochromes, especially CYP3A4, should be carefully monitored or avoided, whenever is possible.

A significant interaction with aprepitant (CYP3A4 inhibitor) is suggested by available phase I data from the PM1183-A-008-13 study. PM01183 clearance was reduced by 50%, approximately, in the presence of aprepitant. Although all patients eventually recovered, the use of aprepitant is currently forbidden in all PM01183 studies.

### Evaluability of Patients

### Phase I

An evaluable patient for the primary objective of this phase I part (i.e., determination of the MTD and RD) should have received at least one complete infusion of atezolizumab and PM01183 and be followed for at least one complete cycle (i.e., three weeks=21 days). Patients who are discontinued early or miss/delay doses and/or clinically relevant assessments (i.e., Hematology and Biochemistry-A) will be evaluable if these events are the consequence of treatment-related toxicity (excluding hypersensitivity reactions and/or extravasations).

### Evaluation Criteria

### Primary Endpoint

### Phase I

### Determination of MTD and RD:

The MTD will be the lowest dose level explored during dose escalation at which more than one third of evaluable patients experience a DLT during Cycle 1.
The RD will be the highest dose level explored during dose escalation at which less than one third of evaluable patients experience a DLT during Cycle 1.

If the DLTs observed with the PM01183 and atezolizumab combination without G-CSF prophylaxis are exclusively related to neutropenia, the MTD and RD will also be determined with Primary G-CSF prophylaxis.

### Secondary Endpoints

Safety: patients will be evaluable for safety if they have received at least one partial infusion of atezolizumab and PM01183. AEs will be graded according to the National Cancer Institute Common Terminology Criteria for Adverse Events (NCI-CTCAE) v. 5.
Efficacy: antitumor activity of the combination will be evaluated in terms of:
   Progression-free survival defined as the time from the date of registration to the date of documented progression per RECIST v. 1.1 or death (regardless of the cause of death). If the patient receives further antitumor therapy or is lost to follow-up before PD, PFS will be censored at the date of last tumor assessment before the date of subsequent antitumor therapy.
   Duration of response (DoR) will be calculated from the date of first documentation of response per RECIST v. 1.1 (complete or partial response, whichever comes first) to the date of documented PD or death. The censoring rules defined above for PFS will be used for DoR.
   Clinical benefit defined as percentage of evaluable patient with complete response, partial response or stable disease lasting ≥3 months, as defined by RECIST v 1.1.
   Overall survival (OS): calculated from the date of registration to the date of death (death event) or last contact (in this case, survival will be censored on that date).
   Mid- and long-term survival (OS at 12, 18 and 24 months) will be the Kaplan-Meier estimates of the probability of being alive at these time points.
Pharmacokinetics: PK parameters will be evaluated in plasma by standard non-compartmental methods (compartmental modeling may be performed if appropriate).
Pharmacogenetics: factors that may help to explain individual variability in main PK parameters, the presence or absence of germline mutations or polymorphisms that may be involved in the metabolism and/or transport of PM01183 will be analyzed in leukocyte DNA extracted.
Pharmacogenomics: In order to determine predictive/prognostic markers of response and/or resistance to PM01183 and atezolizumab, tumor samples available at baseline will be evaluated in all patients. In addition, blood samples (Day 1 of every cycle and end-of-treatment) and on-treatment tumor sample from biopsy (4th to 6th weeks after treatment onset) will be obtained and evaluated for patients consenting to PGx sub-study.

### Results

A total of 26 patients were treated, including male 14 pts (53.8%) and female 12 pts (46.2%) with median age 60.6 years

Five pts received Lurbinectedin 2.5 mg/m²+atezolizumab 1200 mg, and 3 pts were evaluable without DLT.

Out of the 21 pts who received Lurbinectedin 3.2 mg/m²+atezolizumab 1200 mg (6 pts with primary G-CSF, 5 pts (20.8%) developed DTLs:
2 pts G3 febrile neutropenia (9.5%) (1 pt with G4 thrombocytopenia)
2 pts G4 neutropenia lasting more than 72 hours (9.5%)
1 pt G4 thrombocytopenia (4.8%).

Most frequent hematological adverse events ≥grade 2 (21 pts) in DL2 cohort were:
9 pts neutropenia (42.9%)
6 pts thrombocytopenia (28.6%)
4 pts anemia (19.1%)
1 pt lymphopenia (4.8%)
1 pt febrile neutropenia (4.8%)

The most common non-hematological treatment related adverse events ≥grade 2 was asthenia 30.8% (8 pts), Table 1. No treatment-related deaths were reported.

As the proportion of patients that experienced a DLT was less than a third (20.8%), the MTD could not be calculated.

Objective responses (ORR) were observed in 15 pts (57.7%), including complete responses (CR) in 2 pts (7.7%), partial response (PR) in 13 pts (50%). Stable disease (SD) was observed in 7 pts (26.9%) and 3 pts (11.54%) were in progressive disease (PD). Disease control rate (DC) was 84.61%.

With 8 pts censored for progression, median PFS was 4.93 months (range 3.37-7.47 months).

### CONCLUSIONS

The combination of Lurbinectedin plus atezolizumab was well tolerated, without unexpected toxicities.
Transient hematological toxicity was dose limiting.
The RD is Lurbinectedin 3.2 mg/m² on Day 1 plus atezolizumab 1200 mg Day 1 with G-CSF.
Preliminary anti-tumor activity is remarkable.
Remarkable antitumor activity of the combination was observed in more than half of all patients (RR 57.7%) including two complete responses. This activity is currently not expected for this population with approved drugs. This data supports the synergy between the two compounds.

### CLAUSES

1. A method of treatment of cancer, the method comprising administering a combination therapy of lurbinectedin and atezolizumab to a patient in need thereof, wherein lurbinectedin is administered at a dose of between 2.5 to 3.2 mg/m², thereby treating the cancer.
2. The method of clause 1, wherein lurbinectedin is administered at a dose of 2.5 mg/m².
3. The method of clause 1, wherein lurbinectedin is administered at a dose of 2.8 mg/m².
4. The method of clause 1, wherein lurbinectedin is administered at a dose of 3.0 mg/m².
5. The method of clause 1, wherein lurbinectedin is administered at a dose of 3.1 mg/m².
6. The method of clause 1, wherein lurbinectedin is administered at a dose of 3.2 mg/m².
7. The method of clause 1, wherein lurbinectedin is administered at between 2.8 to 3.2 mg/m², or between 3.0 and 3.2 mg/m², or between 3.1 and 3.2 mg/m².
8. The method according to any preceding clause, wherein atezolizumab is administered at a dose of 840 mg to 1680 mg, preferably 900 mg to 1500 mg, 1000 mg to 1400 mg, 1100 mg to 1300 mg or 1200 mg.
9. The method according to clause 8, wherein atezolizumab is administered at a dose of 1200 mg.
10. The method of clause 1, wherein lurbinectedin is administered at a dose of 3.2 mg/m2 and atezolizumab is administered at a dose of 1200 mg.
11. The method according to any preceding clause wherein lurbinectedin and atezolizumab are administered concurrently, separately or sequentially.
12. The method of clause 11 wherein atezolizumab is administered initially, immediately followed by lurbinectedin.
13. The method of any preceding clause, wherein multiple administrations of either lurbinectedin, or atezolizumab, or both, are given.
14. The method of any preceding clause, wherein lurbinectedin is administered by subcutaneous, intravenous or intraperitoneal route; preferably intravenous infusion.
15. The method of any preceding clause, wherein lurbinectedin is administered with an infusion time of up to 24 hours, 1 to 12 hours, 1 to 6 hours and most preferably 1 hour.
16. The method of any preceding clause, wherein atezolizumab is administered by subcutaneous, intravenous, or intraperitoneal route, preferably intravenous infusion.
17. The method of any preceding clause, wherein atezolizumab is administered with an infusion time of 1 hour; or of 30 minutes for the second and subsequent infusions.
18. The method of any preceding clause, having an administration cycle of one every one to four weeks; preferably once every three weeks or every four weeks.
19. The method of any preceding clause, having an administration cycle of once every 21 days.
20. The method of any preceding clause, wherein lurbinectedin is administered on day 1 or a cycle.
21. The method of any preceding clause, wherein atezolizumab is administered on day 1 of a cycle.
22. The method of any preceding clause, wherein the method further comprises administration of granulocyte-colony stimulating factor (G-CSF).
23. The method of clause 22, wherein in Cycle 1, the patient receives primary prophylaxis with G-CSF starting 24-72 hours after Day 1 of Cycle 1, and for five days.
24. The method of clause 23, wherein G-CSF is administered during one or more subsequent cycles.
25. The method according to any preceding clause, wherein the cancer is a solid tumour.
26. The method according to clause 25, wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer, and sarcomas.
27. The method according to clause 26, wherein the lung cancer is mesothelioma, malignant mesothelioma, malignant pleural mesothelioma, malignant peritoneal mesothelioma, preferably malignant pleural mesothelioma.
28. The method according to clause 26, wherein the lung cancer is non-small cell lung cancer.
29. The method according to clause 26, wherein the lung cancer is small cell lung cancer.
30. The method according to clause 29, wherein the small cell lung cancer is extensive stage small cell lung cancer (ES-SCLC).
31. The method according to any preceding clause wherein the patient has progressed, including wherein the patient has progressed from first line therapy.
32. The method according to any preceding clause, wherein the patient has progressed from prior platinum treatment.
33. The method according to any preceding clause, wherein the patient is immunotherapy naive.
34. The method according to any preceding clause, wherein the treatment is second line treatment.
35. A method of treatment of small cell lung cancer, the method comprising administering a combination therapy of lurbinectedin and atezolizumab to a patient in need thereof, thereby treating the small cell lung cancer.
36. The method of clause 35, wherein lurbinectedin is administered at a dose of between 2.5 to 3.2 mg/m².
37. The method of clause 35, wherein lurbinectedin is administered at a dose of 2.5 mg/m².
38. The method of clause 35, wherein lurbinectedin is administered at a dose of 2.8 mg/m².
39. The method of clause 35, wherein lurbinectedin is administered at a dose of 3.0 mg/m².
40. The method of clause 35, wherein lurbinectedin is administered at a dose of 3.1 mg/m².
41. The method of clause 35, wherein lurbinectedin is administered at a dose of 3.2 mg/m².
42. The method of clause 35, wherein lurbinectedin is administered at between 2.8 to 3.2 mg/m², or between 3.0 and 3.2 mg/m², or between 3.1 and 3.2 mg/m².
43. The method according to any one of clauses 35 to 42, wherein atezolizumab is administered at a dose of 1200 mg.
44. The method according to any one of clauses 35 to 43, wherein the small cell lung cancer is extensive stage small cell lung cancer (ES-SCLC).
45. The method according to any one of clauses 35 to 44, wherein the patient has progressed, including wherein the patient has progressed from first line therapy.
46. The method according to any one of clauses 35 to 45, wherein the patient has progressed from prior platinum treatment.
47. The method according to any one of clauses 35 to 46, wherein the patient is immunotherapy naive.
48. The method according to any one of clauses 35 to 47, wherein the treatment is second line treatment.
49. The method according to any one of clauses 35 to 48, wherein lurbinectedin and atezolizumab are administered concurrently, separately or sequentially.
50. The method of clause 49 wherein atezolizumab is administered initially, immediately followed by lurbinectedin.
51. The method of any one of clauses 35 to 50, wherein multiple administrations of either lurbinectedin, or atezolizumab, or both, are given.
52. The method of any one of clauses 35 to 51, wherein lurbinectedin is administered by subcutaneous, intravenous or intraperitoneal route; preferably intravenous infusion.
53. The method of any one of clauses 35 to 52, wherein lurbinectedin is administered with an infusion time of up to 24 hours, 1 to 12 hours, 1 to 6 hours and most preferably 1 hour.
54. The method of any one of clauses 35 to 53, wherein atezolizumab is administered by subcutaneous, intravenous, or intraperitoneal route, preferably intravenous infusion.
55. The method of any one of clauses 35 to 54, wherein atezolizumab is administered with an infusion time of 1 hour; or of 30 minutes for the second and subsequent infusions.
56. The method of any one of clauses 35 to 55, having an administration cycle of one every one to four weeks; preferably once every three weeks.
57. The method of any one of clauses 35 to 56, having an administration cycle of once every 21 days.
58. The method of any one of clauses 35 to 57, wherein lurbinectedin is administered on day 1 or a cycle.
59. The method of any one of clauses 35 to 58, wherein atezolizumab is administered on day 1 of a cycle.
60. The method of any one of clauses 35 to 59, wherein the method further comprises administration of granulocyte-colony stimulating factor (G-CSF).
61. The method of clause 60, wherein in Cycle 1, the patient receives primary prophylaxis with G-CSF starting 24-72 hours after Day 1 of Cycle 1, and during five days.
62. The method of any one of clauses 35 to 61, wherein the treatment results in one or more of: reduction in tumour size; delay in growth of tumour; prolongation of life of the patient; delay in disease progression; remission.
63. Use of lurbinectedin in the manufacture of a medicament for the treatment of cancer, wherein said treatment comprises administering a combination therapy of lurbinectedin and atezolizumab to a patient in need thereof, wherein lurbinectedin is administered at a dose of between 2.5 to 3.2 mg/m².
64. Use of atezolizumab in the manufacture of a medicament for the treatment of cancer, wherein said treatment comprises administering a combination therapy of lurbinectedin and atezolizumab to a patient in need thereof, wherein lurbinectedin is administered at a dose of between 2.5 to 3.2 mg/m².
65. Use of lurbinectedin and atezolizumab in the manufacture of a medicament for the treatment of cancer, wherein said treatment comprises administering a combination therapy of lurbinectedin and atezolizumab to a patient in need thereof, wherein lurbinectedin is administered at a dose of between 2.5 to 3.2 mg/m².
66. Lurbinectedin for use in the treatment of cancer, wherein said treatment comprises administering a combination therapy of lurbinectedin and atezolizumab to a patient in need thereof, wherein lurbinectedin is administered at a dose of between 2.5 to 3.2 mg/m².
67. Atezolizumab for use in the treatment of cancer, wherein said treatment comprises administering a combination therapy of lurbinectedin and atezolizumab to a patient in need thereof, wherein lurbinectedin is administered at a dose of between 2.5 to 3.2 mg/m².
68. Lurbinectedin and atezolizumab for use in the treatment of cancer, wherein said treatment comprises administering a combination therapy of lurbinectedin and atezolizumab to a patient in need thereof, wherein lurbinectedin is administered at a dose of between 2.5 to 3.2 mg/m².
69. Lurbinectedin for use in the treatment of cancer, wherein in said treatment lurbinectedin is administered in combination with atezolizumab to a patient in need thereof, wherein lurbinectedin is administered at a dose of between 2.5 to 3.2 mg/m².
70. Atezolizumab for use in the treatment of cancer, wherein in said treatment atezolizumab is administered in combination with lurbinectedin to a patient in need thereof, wherein lurbinectedin is administered at a dose of between 2.5 to 3.2 mg/m².
71. A pharmaceutical package comprising lurbinectedin and atezolizumab, optionally together with instructions for their combination use, wherein lurbinectedin is administered at a dose of between 2.5 to 3.2 mg/m².
72. Use of lurbinectedin in the manufacture of a medicament for the treatment of small cell lung cancer, wherein said treatment comprises administering a combination therapy of lurbinectedin and atezolizumab to a patient in need thereof.
73. Use of atezolizumab in the manufacture of a medicament for the treatment of small cell lung cancer, wherein said treatment comprises administering a combination therapy of lurbinectedin and atezolizumab to a patient in need thereof.
74. Use of lurbinectedin and atezolizumab in the manufacture of a medicament for the treatment of small cell lung cancer, wherein said treatment comprises administering a combination therapy of lurbinectedin and atezolizumab to a patient in need thereof.
75. Lurbinectedin for use in the treatment of small cell lung cancer, wherein said treatment comprises administering a combination therapy of lurbinectedin and atezolizumab to a patient in need thereof.
76. Atezolizumab for use in the treatment of small cell lung cancer, wherein said treatment comprises administering a combination therapy of lurbinectedin and atezolizumab to a patient in need thereof.
77. Lurbinectedin and atezolizumab for use in the treatment of small cell lung cancer, wherein said treatment comprises administering a combination therapy of lurbinectedin and atezolizumab to a patient in need thereof.
78. Lurbinectedin for use in the treatment of a small cell lung cancer, wherein in said treatment lurbinectedin is administered in combination with atezolizumab to a patient in need thereof.
79. Atezolizumab for use in the treatment of a small cell lung cancer, wherein in said treatment atezolizumab is administered in combination with lurbinectedin to a patient in need thereof.
80. A pharmaceutical package comprising lurbinectedin and atezolizumab, optionally together with instructions for their combination use.
81. A kit comprising atezolizumab, together with instructions for combination use with lurbinectedin according to any preceding claim.

1. Lurbinectedin for use in the treatment of cancer, wherein said treatment comprises administering to a patient in need thereof a combination of lurbinectedin and atezolizumab; wherein lurbinectedin is administered at a dose of 3.2 mg/m² and atezolizumab is administered at a dose of 1200 mg; and wherein lurbinectedin and atezolizumab are administered on day 1 of a 21 day cycle.

2. Lurbinectedin for use according to claim 1 wherein lurbinectedin and atezolizumab are administered concurrently, separately or sequentially; preferably wherein atezolizumab is administered initially, immediately followed by lurbinectedin.

3. Lurbinectedin for use according to any preceding claim, wherein multiple administrations of either lurbinectedin, or atezolizumab, or both, are given.

4. Lurbinectedin for use according to any preceding claim, wherein lurbinectedin is administered by subcutaneous, intravenous or intraperitoneal route; preferably intravenous infusion; optionally wherein lurbinectedin is administered with an infusion time of up to 24 hours, 1 to 12 hours, 1 to 6 hours and most preferably 1 hour.

5. Lurbinectedin for use according to any preceding claim, wherein atezolizumab is administered by subcutaneous, intravenous, or intraperitoneal route, preferably intravenous infusion; optionally wherein atezolizumab is administered with an infusion time of 1 hour; or of 30 minutes for the second and subsequent infusions.

6. Lurbinectedin for use according to any preceding claim, wherein the method further comprises administration of granulocyte-colony stimulating factor (G-CSF).

7. Lurbinectedin for use according to claim 6 when G-CSF is administered on day 1 of a 21 day cycle.

8. Lurbinectedin for use according to claim 7, wherein the patient receives primary prophylaxis with G-CSF starting 24-72 hours after Day 1 of each cycle, and for five days.

9. Lurbinectedin for use according to any preceding claim comprising 1, 2, 3, 4, 5, 6, 7, 8, 9, 10, 11, 12, 13, 14, 15, 16, 17, 18, 19, 21, 22, 23, 24, 25, 26, 27, 28, 29, 30 or more cycles.

10. Lurbinectedin for use according to according to any preceding claim, wherein the cancer is a solid tumour; preferably wherein the solid tumour is selected from the group consisting of prostate cancer, breast cancer, lung cancer, colorectal cancer, melanomas, bladder cancer, brain/CNS cancer, cervical cancer, oesophageal cancer, gastric cancer, head/neck cancer, kidney cancer, liver cancer, lymphomas, ovarian cancer, pancreatic cancer, and sarcomas.

11. Lurbinectedin for use according to according to claim 10, wherein the lung cancer is mesothelioma, malignant mesothelioma, malignant pleural mesothelioma, malignant peritoneal mesothelioma, preferably malignant pleural mesothelioma;

12. Lurbinectedin for use according to according to claim 10, wherein the lung cancer is non-small cell lung cancer or wherein the lung cancer is small cell lung cancer; optionally extensive stage small cell lung cancer (ES-SCLC).

13. Lurbinectedin for use in the treatment of small cell lung cancer, wherein said treatment comprises administering to a patient in need thereof a combination of lurbinectedin and atezolizumab; optionally wherein the small cell lung cancer is extensive stage small cell lung cancer (ES-SCLC).

14. Lurbinectedin for use according to according to claim 13, wherein lurbinectedin and atezolizumab are administered according to the regimen of any one of claims 1 to 9.

15. Lurbinectedin for use according to any preceding claim wherein the patient has progressed;
optionally wherein the patient has progressed from first line therapy; and/or
optionally wherein the patient has progressed from prior platinum treatment; and/or
optionally wherein the patient is immunotherapy naive.

16. Lurbinectedin for use according to any preceding claim, wherein the treatment is second line treatment.

17. Lurbinectedin for use according to any preceding claim:
wherein the patient has progressed from prior immunotherapy; and/or
wherein the patient has progressed from receiving standard of care; and/or
wherein the patient has progressed from receiving standard of care which includes immunotherapy.

18. Lurbinectedin for use according to any preceding claim:
wherein the patient has progressed from platinum-etoposide, e.g. cisplatin-etoposide or carboplatin-etoposide; carboplatin-oral topotecan; cisplatin-irinotecan; or carboplatin-gemcitabine; and/or
wherein the patient has progressed from an immune checkpoint inhibitor together with a platinum agent and etoposide; optionally carboplatin-etoposide-atezolizumab, or platinum-etoposide-durvalumab; and/or
wherein the patient has progressed from nivolumab-ipilimumab.

19. Lurbinectedin for use according to any preceding claim, wherein the treatment results in one or more of: reduction in tumour size; delay in growth of tumour; prolongation of life of the patient; delay in disease progression; remission.

20. A method of inhibiting cancer cell growth, comprising contacting cancer cells with a combination of lurbinectedin and atezolizumab; wherein said cancer cells are small cell lung cancer cells; optionally extensive stage small cell lung cancer (ES-SCLC) cells; or wherein the cancer cells are mesothelioma, optionally malignant plural mesothelioma.

21. A kit comprising lurbinectedin, together with instructions for combination use with atezolizumab according to any preceding claim.
